SEQUENCE LISTING (21 sequences)

SEQ ID NO: 1 - protein, 227 aa, Bacillus sp.
  PEPTIDE 22..227
MKRFLFLTLLLCMMFVLGACGSDNGNSNNNANGISDRDSNMEKSDERSSVDEGLNQEGNSANLGVEQNNPQQKNEGGVELVTVDLKDTEGKKVGTVNLEEIDAGIRIDLEASNLPPGTHGFHIHETASCVAPTFESAGGHFNPTNASHGVDNEGGPHAGDLPNIEVGKDGTIKEEITAKNVTLKTHKENSLFDSDGSALVIHAKADDNKSQPSGNAGDRIACSEIGE

SEQ ID NO: 2 - DNA, 684 bp, Bacillus sp.
  gene 1..684
  source 1..684: mol_type = genomic DNA
atgaaacgattcctttttttaacgcttcttttgtgcatgatgtttgtacttggggcatgcggaagtgacaatggcaatagtaataataacgcgaatggtattagcgatagagatagcaacatggaaaaaagtgacgagaggtcttcggtagatgaagggttaaatcaagaagggaattcagcaaatttgggcgtggagcaaaataatcctcaacaaaagaatgaaggtggggtagagttagtgactgttgatttgaaggatacggaaggcaagaaagtgggaacggtaaacctcgaagaaatcgatgccgggattcgtatcgatttggaggcatcgaatttgccaccgggaacacatggttttcatattcatgaaacagcatcatgcgttgctccaacttttgaatcagcaggcggtcattttaaccctacaaacgcgagccacggtgttgacaatgaaggaggaccacatgcaggcgaccttcccaatattgaagtaggaaaagatggtacaattaaagaagaaataacagctaaaaatgtcactttaaagacccacaaagaaaattcattgttcgattcagatggatcggcgcttgtgattcatgccaaagcggacgacaataagtctcagccgtccggtaatgcaggagatcgcatcgcatgtagtgagattggtgaatga

SEQ ID NO: 3 - protein, 179 aa, Bacillus sp.
  PEPTIDE 27..179
MKKRYFLSALLTGMVLFSGQTLNAGHAEKKQMPVKITMINQNGKVSGYATLTQTQMGLNVEVNVRGLKPGVHGIHFHENGSCIAPTFDSAGGHFNPEHKEHGLKNPMGPHAGDLKNVVADRNGVLQTNFMTSRVTLQKGVKDSLRDVNSSALVIHAQEDDQKTNPAGNSGPRVMCGVIK

SEQ ID NO: 4 - DNA, 540 bp, Bacillus sp.
  gene 1..540
  source 1..540: mol_type = genomic DNA
atgaaaaagcgctattttttatccgctttgttaacaggaatggttttgttctccgggcaaacgctaaatgcaggccatgctgaaaagaaacagatgcctgtcaaaatcactatgatcaatcaaaatggaaaagtctcagggtatgcgacactcactcaaacccagatgggcctgaacgttgaggtcaatgtacgtgggttaaagcctggtgttcatggcattcattttcatgaaaatggttcatgcattgctccaacattcgattcagctggaggccactttaatcccgaacataaggagcacggccttaaaaatccaatgggtccacatgccggcgatttaaaaaatgtggtcgcggaccgtaacggggttctgcaaaccaactttatgacttcaagagtcacccttcaaaaaggtgtgaaggacagtttgcgtgatgtgaatagctccgcattagtcattcacgcacaagaagatgaccagaaaacaaaccccgctggcaattcaggcccacgcgtcatgtgcggcgtgattaaatag

SEQ ID NO: 5 - protein, 296 aa, Bacillus sp.
  PEPTIDE 1..296
MDSERKTKQYLMEVQDWLQNVHDFLNEQEAEADPDFLAEIERVKNDVKNQLAVETISESVISELQGTVQRMNNRVVELYERNQTRIRESVPIGKHVLPPLPYEYNALEPYISEEIMRLHHTKHHQSYVDGLNKAEEKMKQARKRNDFDLIKHWEREAAFHGSGHYLHCIFWEIMNPKGGGKPKGELLAQIEKHFGSYELFKKHFSEAAKKVEGVGWAILVWSPRSRKLEILQAERHQFLTQWDTIPILVLDVWEHAYYLQYKNDKGTYVDQWWNVVYWPNAEKRFAEAKKLKWEPY

SEQ ID NO: 6 - DNA, 891 bp, Bacillus sp.
  gene 1..891
  source 1..891: mol_type = genomic DNA
atggattctgaaagaaaaacgaaacaatatttaatggaagtgcaagactggctgcaaaacgttcatgattttctgaatgagcaagaggcggaagcggatccagattttttggccgagattgagcgtgtaaaaaatgatgtgaagaatcaacttgcggtagaaaccataagtgagtcggttatatccgaacttcaaggtaccgttcagcgaatgaacaatcgtgtcgtcgaattatatgaaagaaatcaaacaagaattcgtgaatccgtgccgatagggaaacatgtacttccgccattaccatatgaatacaacgccttagaaccttatattagcgaagaaatcatgaggctccaccatacaaaacatcatcaaagttatgtagatgggctaaataaagccgaagaaaaaatgaagcaagcaagaaaaaggaatgattttgatttaattaaacattgggaacgagaagccgcctttcacgggtcgggacattatctgcattgtatcttttgggaaattatgaatcctaaaggcggtggaaaaccaaaaggagagcttcttgctcaaatcgaaaaacattttggcagctatgagttatttaaaaagcatttttcagaagcagctaaaaaagttgaaggggtaggatgggctattttagtttggtcgccccgatcgcgaaaactggaaattttgcaagcagagcgccatcaatttttaacacagtgggataccatcccgattcttgttctggatgtttgggagcatgcctattacttgcaatataaaaatgataaaggaacgtatgtagatcaatggtggaacgtagtttattggccaaatgcagaaaaaagatttgccgaagcaaaaaaactcaaatgggaaccttattaa

SEQ ID NO: 7 - protein, 205 aa, Bacillus sp.
  PEPTIDE 23..205
MSKMYHYLLLIVIGLLLTSACAEKQVMEFGEQPLMLDDVEAKADIYDKEGEMMGDVVFSFIDDKVLIQADVQNLPPGWHGFHIHENGSCGLTEQGADFSKAGGHFHHADDGQHGYHAGDMPPLYVNENGHAYMTVLMDRFSLDEVLSGNGTSVIIHEEPDNLGHIPERYQSVKSDQPGPDEQTLATGDGGSRLACGEIKRVEKEE

SEQ ID NO: 8 - DNA, 618 bp, Bacillus sp.
  gene 1..618
  source 1..618: mol_type = genomic DNA
atgagtaaaatgtatcattaccttcttctgattgtcatcggcttattattgacaagtgcatgcgcagagaagcaagtaatggagtttggtgaacaaccgcttatgcttgacgatgtggaagcgaaagcggacatatacgataaagaaggagaaatgatgggagatgtcgttttttcatttattgatgataaggttctcatccaagccgatgttcaaaatttgcccccaggatggcatgggttccatattcatgaaaatggttcttgtggcttgactgaacaaggagccgatttttccaaagcaggaggtcactttcatcatgcagacgatggacagcatggctatcatgcaggagatatgccaccgctgtacgtcaacgaaaatggtcatgcttatatgacggttcttatggaccgcttttcattagatgaagtgctaagtggcaatggaacgtcagtcatcattcacgaggaaccagataatcttggtcatatcccagaacgttatcaatctgttaaaagcgatcagccaggaccagatgaacaaacactagctacgggagatgggggtagtcgtttagcttgtggagaaattaaacgcgtagagaaagaagaataa

SEQ ID NO: 9 - protein, 261 aa, Variovorax boronicumulans
  PEPTIDE 21..261
MQRFIVSLAVAGFVAGCAQTGPMSAQSMTPAAPMAPAPMAAPMAPPPPPATPPAAQVFRQAPLPYAADALEPVIDKATMEIHHGRHHKAYYDALNNAAASSPEVARSTLEQLVATASRQTMVVRNNAGGAWNHAFFWNTMAPAGQRGAPSPALMARVQADFGSMETLMRQFNQAGASRFGSGWAWLIVKDGKLAVSSTPNQDNPLMDVAEVRGTPILGNDVWEHAYYLKYQNRRADYLAAWWQVVNWNEVNRRFAAATAAR

SEQ ID NO: 10 - DNA, 786 bp, Variovorax boronicumulans
  gene 1..786
  source 1..786: mol_type = genomic DNA
atgcagagattcatcgtgtcgctcgcggtcgcgggtttcgtcgccggatgcgcgcagaccggccccatgtcggcccagtccatgacgcccgccgctcccatggcgcccgccccgatggccgcgcccatggcaccgccgccaccgcccgcgaccccgcccgccgcccaggtgttccgccaggcgccgttgccctatgcggccgacgcgctggagccggtgatcgacaaggcgacgatggagatccatcacggccgccatcacaaggcctactacgacgcgttgaacaacgccgccgcctcgagccccgaggtcgcgcgctcgaccctcgaacagctcgtcgccaccgcatcgcgccagacgatggtcgtgcgcaacaacgccggcggcgcctggaaccatgccttcttctggaacaccatggcaccggccggccagcgcggcgcgccgtcgcccgcgctgatggcgcgcgtccaggccgacttcggctccatggagacgctgatgcgccagttcaaccaggccggcgcctcgcgcttcggttccggctgggcctggctgatcgtcaaggacggcaagctcgccgtcagctccacgcccaaccaggacaacccattgatggacgtggccgaagtgcgtggcacgcccatcctgggcaacgacgtctgggagcatgcctactacctgaagtaccagaaccgtcgcgccgactatctcgccgcctggtggcaggtggtgaactggaacgaagtcaaccgccgcttcgccgccgcgaccgccgcgcgctga

SEQ ID NO: 11 - protein, 174 aa, Bacillus sp.
  PEPTIDE 1..174
MNYYQYGYPAYSYYPYRNMPAMAYAIIQGGPLAPKLNGYIFFREVPNGVEAFIEINGLPQYQKGSGDNKPVGPHGFHLHEKGVCEVGDEEDPFQSAGGHWNPDNQPHGNHAGDFPVLFSNDGYSKMSFFTNRFKVDDVIGKGVIIHQNPDDYRSQPSGDAGKRLGCGVITKYGM

SEQ ID NO: 12 - DNA, 525 bp, Bacillus sp.
  gene 1..525
  source 1..525: mol_type = genomic DNA
atgaattattatcaatacggctatcctgcctattcatattatccttatagaaatatgcctgcgatggcatatgccatcattcagggaggcccactcgctcccaagttaaacggatatatcttctttcgggaagttccgaatggagtagaagcattcatagagataaatggactgccgcagtatcaaaaaggaagcggtgataataagccagttgggccgcatggctttcatcttcatgaaaaaggagtatgtgaagtcggagacgaggaggatccttttcaatctgcaggaggacattggaatccggataatcagccgcatggaaatcacgcaggagattttccggtactgttctcaaacgatggctacagtaaaatgtctttttttacgaacaggttcaaagtggatgatgtcatcggaaaaggagtgatcatccaccaaaatccggatgactatagatcccagccttccggggatgcagggaagaggctcggctgtggagtgattacgaagtatggaatgtag

SEQ ID NO: 13 - protein, 245 aa, Pedobacter nyackensis
  PEPTIDE 36..245
METNTRRDFIKTTLTASLAVVIGTPVLLSGGTALAAAVTAEEGTGFAALKFAQVPLKYAFNALEPNIDALTMDIHYTKHHAAYVKNVNDAIAAEKIPYKTEAEFFNNASKLSAKARNNGGGAWNHNFFFETLKPGGATGPEGKLKDAIVKSFVSVDKFKEQFSAAAASRFGSGWAWLVNDKGTLKICSTANQDNPLFDNAEVKGVPLLGLDVWEHAYYLKYQNKRADYIANWWNVVDWDVVSKRL

SEQ ID NO: 14 - DNA, 738 bp, Pedobacter nyackensis
  gene 1..738
  source 1..738: mol_type = genomic DNA
atggaaacaaacacccgaagagactttattaaaactacgttgacggcctcattggcagttgttataggtaccccagttttgttaagcggagggactgcattggccgctgctgtaactgctgaagagggcacgggctttgctgcattgaagtttgcgcaagtgcctctaaaatatgcttttaatgccttggagcctaatatagatgcgcttactatggacattcattatacaaaacaccatgcggcctatgttaaaaatgtaaatgatgcaattgcggccgaaaagattccgtacaaaacagaggcggaattttttaataatgcttcgaagctttcggccaaagcacgtaacaatggtggtggggcatggaatcataatttcttttttgagactttaaagccagggggggcaactggaccagaaggaaaattaaaagatgctattgtgaagtcatttgtttcggttgataaatttaaagaacaattttctgctgctgcggcatcacgttttggttcaggatgggcatggttggttaatgataagggaactttaaagatttgttcgaccgctaatcaggataatcctttatttgataatgcggaagtgaaaggcgtacctttgttgggacttgatgtatgggagcacgcttattatcttaagtatcagaataaacgtgcagattatattgccaactggtggaatgtcgtggattgggatgtggtgagtaaaaggctgtag

SEQ ID NO: 15 - protein, 255 aa, Mongoliicoccus sp.
  PEPTIDE 29..255
MSNFSRRSFLTRTTKATLAVGIGSSALGSTLLACNNATTRQDEKAVLDNLRTDFTQTDLPYDYSALEPHIDARTMEIHYSRHAKSYAENLREAAEEEDVDTSKPVEELMMNISHYSDKMRNNGGGHYNHELFWKIMSPDGGGQPQGELAQAINNSFGSYDEFVEKFEQEAKDRFGSGWAWLVLDRDKNLAVGSTPNQDNPLMNDVDFQGIPLMGIDVWEHAYYLKYQNERGEYISNWWNVVDWQTIARRYEKLRA

SEQ ID NO: 16 - DNA, 768 bp, Mongoliicoccus sp.
  gene 1..768
  source 1..768: mol_type = genomic DNA
atgagcaatttttcaagaaggagcttccttacccgcaccaccaaagccacccttgccgtaggcataggctcctcagcactcggatccaccctgctggcatgcaacaatgccaccacccggcaggatgaaaaagcggtgctggacaatctcaggacggactttacccaaactgatcttccctatgactacagtgctttggagccccatatcgacgcccgtacaatggagatccactactccagacatgccaaaagttatgcagagaacctaagggaagcagcggaggaggaagacgtggacacttctaagccggtggaagaactgatgatgaacatcagccattactcggacaagatgagaaacaacgggggaggccattacaaccatgagcttttctggaagatcatgtctcccgacggcggtggccaaccgcagggggagcttgcccaggccatcaacaattccttcggcagctatgatgaattcgtggaaaaattcgaacaggaagccaaggaccgcttcggttcgggctgggcttggctcgtgctggacagggacaaaaacctggccgtgggctccaccccaaaccaggacaatccgctgatgaatgacgtggatttccagggcattcccctgatgggtatagatgtttgggaacatgcttattacctcaaatatcagaacgaacggggggagtacatttccaactggtggaatgtcgtcgactggcaaaccatagcccggagatacgaaaaattaagagcttaa

SEQ ID NO: 17 - protein, 249 aa, Arcicella aquatica
  PEPTIDE 27..249
MNRKSFLKTTLASAFALSSLNSFSFSLRRSYNDKLPKLTLEEVMLESAPFSLAALPYAPDSLEPSIDKLTMEIHHDRHHKAYVDNLNKAVAGTPYEKLSLWDLLKEAGKAPAAIRNNAGGHWNHTFFWNVMSPKAGGSPKGELLDEITKTFGSFDKFKEEFAKAGTTRFGSGWAWLIVQDKKLVITSTPNQDNPLMDVAEKKGSPILALDVWEHAYYLKYQNKRADYITAFWNVVNWDAVSKNFENLKK

SEQ ID NO: 18 - DNA, 750 bp, Arcicella aquatica
  gene 1..750
  source 1..750: mol_type = genomic DNA
atgaaccgtaaaagttttttaaagacaacattagcatcggcttttgcattgtctagcttaaattcattcagtttttcattaagaagaagctataatgacaaattacctaaattgacattagaagaagtaatgcttgaatcagcgcctttctctttagcagcgttgccttatgcacctgatagtttagagccgagcattgataagttgacgatggagattcaccatgatcgccatcacaaagcatatgttgataatttgaataaagctgtggctggtactccttacgaaaaattatctttgtgggatttattgaaagaagcaggaaaagcaccagctgctattagaaataatgcaggtggacattggaatcatacttttttctggaatgtaatgtcgcctaaagctggaggttctcccaaaggtgaattattggatgaaatcactaaaacctttggttcgtttgataaattcaaagaagagtttgctaaagctggaactactcgctttggttcaggttgggcttggttaattgttcaagataagaaattagtgattacttcaacgccaaatcaagataaccctttgatggatgtggctgaaaagaaaggttcgccaattttagccttagatgtttgggaacacgcttattaccttaaataccaaaacaaacgtgcggattatatcactgcgttctggaatgtagtaaattgggatgctgtttcaaagaattttgaaaacttgaagaaataa

SEQ ID NO: 19 - protein, 251 aa, Paraburkholderia sediminicola
  PEPTIDE 31..251
MLNTLISRRHLMAAGSMASFSVLFSKLAYGAATGDAGPGASTATTVVPAFLGSSPQTLPPLPWADNALEPTISARTIGIHYGKHHRAYFDNLHKLLAGTPLEQASLEQIIMQSHDQPALADVFNNAAQAWNHNFYWNSLSPAPSAPSEKLQAAITRKFGSVDALAKALVATSASQFGSGWGWLVLDRGELAVVKTSNAETPFTRGLVPLLTVDVWEHAYYLDYQNRRPDYLVATVSRHLNWAFASANLDRG

SEQ ID NO: 20 - DNA, 756 bp, Paraburkholderia sediminicola
  gene 1..756
  source 1..756: mol_type = genomic DNA
atgctcaacacccttatttcccggcggcatctgatggccgcgggatccatggcctcattcagcgtgctgttttcgaagcttgcctacggcgcggccacaggcgacgccgggcccggcgcaagcacggcgacgaccgtcgtcccggcatttctcggctcgtcgccgcaaacactgccgccgctgccatgggctgacaatgcgcttgagccgaccatttcggcgcgcaccatcggcattcattacggcaagcaccaccgggcctatttcgacaatctgcacaagcttcttgccggcacgccgctcgaacaggcatcgctcgaacagatcatcatgcagtcgcatgaccagcccgcgctcgcagacgttttcaacaacgccgcgcaggcatggaaccacaacttctactggaactcgctgagcccggcgccgagcgcgccgagcgagaaacttcaggcggcgatcacccgcaagttcggttccgtcgatgcactcgcgaaggcgctcgtcgcaacgtcggcatcgcagttcggcagtggctggggatggctcgtgctcgatcgcggagaactcgcggtcgtcaagacgagcaacgcggaaacgccgtttacgcgtggactcgtaccgctgctaaccgtggatgtatgggagcacgcgtactacctggactatcagaaccgtcgaccggactatctggtcgcgacggtctcgcgtcacctgaactgggcgttcgcctcagccaatcttgaccgtggctga

SEQ ID NO: 21 - protein, 202 aa, Bacillus licheniformis
  PEPTIDE 1..202
MAYKLPELPYAYDALEPHIDKETMNIHHTKHHNTYVTKLNEAVAGKQDLESKSVEELVANLDAVPENIRTAVRNNGGGHANHSLFWKLLSPNGGGAPTGELAEAINSKFGSFDQFKEDFAAAAAGRFGSGWAWLVVNNGELEITSTPNQDSPLSEGKTPILGLDVWEHAYYLNYQNRRPDYIKAFWNVVNWDEVARLYSEAK

(19) United States
(12) Patent Application Publication
(10) Pub. No.: US 2026/0085297 A1
(43) Pub. Date: Mar. 26, 2026

(54) BACTERIAL SUPEROXIDE DISMUTASES

(71) Applicant: Novozymes A/S, Bagsvaerd (DK)

(72) Inventors: Morten GJERMANSEN, Greve (DK); Dorotea Raventos Segura, Rungsted (DK)

(73) Assignee: Novozymes A/S, Bagsvaerd (DK)

(21) Appl. No.: 19/315,377

(22) Filed: Aug. 29, 2025

Related U.S. Application Data
(63) Continuation of application No. 18/029,582, now Pat. No. 12,421,504.

(30) Foreign Application Priority Data
Oct. 7, 2020 (EP) 20200528.6

(51) Int. Cl. C12N 9/02; A23K 20/189

(52) U.S. Cl. CPC C12N 9/0089; A23K 20/189; C12Y 115/01001

(57) ABSTRACT

Polypeptides with superoxide dismutase activity of bacterial origin are useful in animal feed additives for improvement of animal growth and performance and in improving animal health, particularly animals exposed to environmental or heat stress.

### CROSS-REFERENCE TO RELATED APPLICATIONS

This application is a continuation of U.S. patent application Ser. No. 18/029,582, filed Mar. 30 2023, which is a national stage entry of PCT/EP2021/077790, filed Oct. 7, 2021, which claims to European Patent Application No. 20200528.6, filed Oct. 7, 2020. The contents of the aforementioned applications are fully incorporated herein by reference.

### REFERENCE TO SEQUENCE LISTING

This application contains a Sequence Listing in computer readable form. The computer readable form is incorporated herein by reference.

### FIELD OF THE INVENTION

The present invention also relates to new polypeptides of bacterial origin having superoxide dismutase activity and to animal feed additive or animal feed comprising superoxide dismutase of bacterial origin.

### BACKGROUND OF THE INVENTION

Superoxide dismutase (SOD, EC 1.15.1.1) is an enzyme that alternately catalyzes the dismutation (or partitioning) of the superoxide (O₂⁻) radical into either ordinary molecular oxygen (O₂) or hydrogen peroxide (H₂O₂). Superoxide is produced as a by-product of oxygen metabolism and, if not regulated, causes many types of cell damage. Thus, SOD is an important antioxidant defense in nearly all living cells exposed to oxygen. SODs are used in the pharmaceutical, cosmetic, food, and environmental protection industries due to their excellent antioxidant properties. Historically, SODs have been isolated from animal or plant sources, but the microbial sources organisms can be easily induced and cultivated on a large scale.

SODs naturally occur in many organisms such as plants, insects, birds, reptiles and mammals. Four types of SODs have been reported according to their metal cofactors: manganese SOD (Mn-SOD), iron SOD (Fe-SOD), copper/zinc SOD (Cu/Zn-SOD), and nickel SOD (Ni-SOD)₂.

Commercially available mammalian (bovine) SOD (Sigma), a Cu/Zn SOD, although having a high activity at neutral pH or at its optimal pH of 7.8, was found to lose 65% of its activity under gastric stability studies. According to its datasheet, its pH range is 7.6-10.5. Commercially available bacterial (from E. coli) Mn-SOD (S5639 from Sigma) was found to lose 57% of its activity under gastric stability studies.

Surprisingly, the inventors of the present invention found that SODs of bacterial origin that are highly active and provide beneficial effects when used in an animal feed or animal feed additive, alone or in combination with a catalase, to improve animal performance and/or health.

### SUMMARY OF THE INVENTION

An aspect of the invention is directed to an isolated polypeptide having superoxide dismutase activity selected from the group consisting of:
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 17;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 19; and
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21.

A further aspect of the invention is directed to an isolated polypeptide of having superoxide dismutase activity selected from the group consisting of
a. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 2;
b. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 4;
c. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 6;
d. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 8;
e. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 10;
f. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 12;
g. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 14;
h. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 16;
i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 18; and
j. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 20.

A further aspect of the invention is directed to an isolated polynucleotide of bacterial origin encoding for a polypeptide having superoxide dismutase activity, wherein the polynucleotide is selected from the group consisting of
a. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 2;
b. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 4;
c. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 6;
d. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 8;
e. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 10;
f. polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 12;
g. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 14;
h. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 16;
i. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 18; and
j. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 20.

Relevant aspects of the invention are directed to a nucleic acid construct or expression vector comprising the polynucleotide as defined above and to a recombinant host cell transformed with the polynucleotide as defined above. In related aspects of the invention, a method of producing a polypeptide having superoxide dismutase activity, comprising: cultivating the host cell defined above under conditions suitable for expression of the polypeptide; and recovering the polypeptide is herein described.

An important aspect of the invention is directed to an animal feed additive comprising a polypeptide of bacterial origin having superoxide dismutase activity, preferably from a Bacillus species, a Neobacillus species, an Alkalihalobacillus species, a Pedobacter species, a Novosphingobium species, a Mongoliicoccus species, a Variovorax species, a Paraburkholderia species and a Arcicella aquatica species.

Alternatively, defined an animal feed additive comprises at least one and no more than two enzyme classes, wherein the at least one enzyme class is a superoxide dismutase of bacterial origin and the optional second enzyme class is a catalase.

An alternate aspect of the invention is directed to a method of improving one or more performance parameters in an animal comprising administering to the animal an animal feed or animal feed additive comprising one or more polypeptides having superoxide dismutase (SOD) activity of bacterial origin, wherein the one or more performance parameters is selected from the group consisting of the European Production Efficiency Factor (EPEF), Feed Conversion Ratio (FCR), Growth Rate (GR), Body Weight Gain (WG), Mortality Rate (MR) and Flock Uniformity (FU).

A method of improving or enhancing immune response and/or reducing inflammation and/or for the modulation of the gut flora in an animal comprising administering to the animal an animal feed or animal feed additive comprising one or more polypeptides having superoxide dismutase activity of bacterial origin is described herein.

A method of reducing or eliminating the use of antibiotics administered to an animal feed, comprising administering to the animal an animal feed or animal feed additive comprising of one or more polypeptides having superoxide dismutase activity of bacterial origin is described herein.

A further aspect of the invention is directed to use of an animal feed additive defined herein as an antioxidant, preferably in feed and feed premixes. Similarly, use of an animal feed additive defined herein for replacing or partially replacing antibiotics in animal feed is described herein.

An additional aspect is directed to an animal feed comprising one or more protein sources and one or more energy sources characterized in that the animal feed further comprises one or more polypeptides of bacterial origin having superoxide dismutase (SOD) activity, wherein the animal feed
a. improves or enhances immune response; or
b. reduces inflammation;
when fed to the animal as compared to animals fed a feed composition without the polypeptides; and
said feed optionally further comprising one or more polypeptides having catalase activity.

A granule, which comprises:
a. a core comprising the polypeptide of the invention, and, optionally
b. a coating consisting of one or more layer(s) surrounding the core is described herein.

An alternative aspect of the invention is a granule, which comprises:
a. a core, and
b. a coating consisting of one or more layer(s) surrounding the core, wherein the coating comprises the polypeptide of the invention.

A further aspect of the invention is directed to an animal feed comprising one or more protein sources and one or more energy sources characterized in that the animal feed further comprises one or more polypeptides of bacterial origin having superoxide dismutase (SOD) activity, wherein said polypeptide is as defined herein.

### OVERVIEW OF SEQUENCE LISTING

SEQ ID NO:1 is the amino acid sequence of a mature polypeptide comprising 227 amino acid residues from Bacillus sp-62775 having superoxide dismutase (SOD) activity. Residues 1 to 21 represent the signal peptide.
SEQ ID NO:2 is the nucleotide sequence comprising 684 residues coding for the polypeptide having superoxide dismutase (SOD) activity of SEQ ID NO:1 from Bacillus sp-62775.
SEQ ID NO:3 is the amino acid sequence of a mature polypeptide comprising 179 amino acid residues from Bacillus sp-18780 having superoxide dismutase (SOD) activity. Residues 1 to 26 represent the signal peptide.
SEQ ID NO:4 is the nucleotide sequence comprising 540 residues coding for the polypeptide having superoxide dismutase (SOD) activity of SEQ ID NO:3 from Bacillus sp-18780.
SEQ ID NO:5 is the amino acid sequence of a mature polypeptide comprising 296 amino acid residues from Bacillus sp-17964 having superoxide dismutase (SOD) activity.
SEQ ID NO:6 is the nucleotide sequence comprising 891 residues coding for the polypeptide having superoxide dismutase (SOD) activity of SEQ ID NO:5 from Bacillus sp-17964.
SEQ ID NO:7 is the amino acid sequence of a mature polypeptide comprising 205 amino acid residues from Bacillus sp-62802 having superoxide dismutase (SOD) activity. Residues 1 to 22 represent the signal peptide.
SEQ ID NO:8 is the nucleotide sequence comprising 618 residues coding for the polypeptide having superoxide dismutase (SOD) activity of SEQ ID NO:7 from Bacillus sp-62802.
SEQ ID NO:9 is the amino acid sequence of a mature polypeptide comprising 261 amino acid residues from Variovorax boronicumulans having superoxide dismutase (SOD) activity. Residues 1 to 20 represent the signal peptide.
SEQ ID NO:10 is the nucleotide sequence comprising 786 residues coding for the polypeptide having superoxide dismutase (SOD) activity of SEQ ID NO:9 from Variovorax boronicumulans.
SEQ ID NO:11 is the amino acid sequence of a mature polypeptide comprising 174 amino acid residues from Bacillus sp-18318 having superoxide dismutase (SOD) activity.
SEQ ID NO:12 is the nucleotide sequence comprising 525 residues coding for the polypeptide having superoxide dismutase (SOD) activity of SEQ ID NO:11 from Bacillus sp-18318.
SEQ ID NO:13 is the amino acid sequence of a mature polypeptide comprising 245 amino acid residues from Pedobacter nyackensis having superoxide dismutase (SOD) activity. Residues 1 to 35 represent the signal peptide.
SEQ ID NO:14 is the nucleotide sequence comprising 738 residues coding for the polypeptide having superoxide dismutase (SOD) activity of SEQ ID NO:13 from Pedobacter nyackensis.
SEQ ID NO:15 is the amino acid sequence of a mature polypeptide comprising 255 amino acid residues from Mongoliicoccus sp-62519 having superoxide dismutase (SOD) activity. Residues 1 to 28 represent the signal peptide.
SEQ ID NO:16 is the nucleotide sequence comprising 768 residues coding for the polypeptide having superoxide dismutase (SOD) activity of SEQ ID NO:15 from Mongoliicoccus sp-62519.
SEQ ID NO:17 is the amino acid sequence of a mature polypeptide comprising 249 amino acid residues from Arcicella aquatica having superoxide dismutase (SOD) activity. Residues 1 to 26 represent the signal peptide.
SEQ ID NO:18 is the nucleotide sequence comprising 750 residues coding for the polypeptide having superoxide dismutase (SOD) activity of SEQ ID NO:17 from Arcicella aquatica.
SEQ ID NO:19 is the amino acid sequence of a mature polypeptide comprising 251 amino acid residues from Paraburkholderia sediminicola having superoxide dismutase (SOD) activity. Residues 1 to 30 represent the signal peptide.
SEQ ID NO:20 is the nucleotide sequence comprising 756 residues coding for the polypeptide having superoxide dismutase (SOD) activity of SEQ ID NO:19 from Paraburkholderia sediminicola.
SEQ ID NO:21 is the amino acid sequence of a mature polypeptide comprising 202 amino acid residues from Bacillus licheniformis having superoxide dismutase (SOD) activity.

### DESCRIPTION OF THE INVENTION

The present invention relates to new polypeptides of bacterial origin having superoxide dismutase activity and to animal feed additive or animal feed comprising superoxide dismutase of bacterial origin.

The present invention also relates to the use of polypeptides of bacterial origin having superoxide dismutase activity in an animal feed or animal feed additive for improving any one of the performance parameter selected from the group consisting of
improving one or more performance parameters, wherein the one or more performance parameters is selected from the group consisting of the European Production Efficiency Factor (EPEF), Feed Conversion Ratio (FCR), Growth Rate (GR), Body Weight Gain (WG), Mortality Rate (MR) and Flock Uniformity (FU),
improving or enhancing immune response and/or reducing inflammation and/or for the modulation of the gut flora in an animal, and
reducing or eliminating the use of antibiotics administered to animal feed.

### Definitions

Animal: The term “animal” refers to any animal except humans. Examples of animals are monogastric animals, including but not limited to pigs or swine (including, but not limited to, piglets, growing pigs, and sows); poultry such as turkeys, ducks, quail, guinea fowl, geese, pigeons (including squabs) and chicken (including but not limited to broiler chickens (referred to herein as broiles), chicks, layer hens (referred to herein as layers)); pets such as cats and dogs; horses (including but not limited to hotbloods, coldbloods and warm bloods) crustaceans (including but not limited to shrimps and prawns) and fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish).

Animal feed: The term “animal feed” refers to any compound, preparation, or mixture suitable for, or intended for intake by an animal. Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Bacterial origin: The term “bacterial origin” is intended to mean obtained, obtainable from or originating from a Bacillus species, a Neobacillus species, an Alkalihalobacillus species, a Pedobacter species, a Novosphingobium species, a Mongoliicoccus species, a Variovorax species, a Paraburkholderia species and a Arcicella aquatica species, most preferably a Bacillus species. Suitably, the said bacterial origin is selected from the group consisting of a Bacillus subtilis, Bacillus licheniformis, Bacillus cereus, Bacillus circulans, Bacillus coagulans, Bacillus pseudomycoides, Bacillus lentus, Neobacillus bataviensis, Alkalihalobacillus hwajinpoensis, Arcicella aquatica, Paraburkholderia sediminicola, Pedobacter nyackensis, Variovorax boronicumulans, Mongoliicoccus sp, Novosphingobium sp, and Pedobacter soli, preferably Bacillus subtilis, Bacillus licheniformis, Bacillus cereus, Bacillus circulans, Bacillus coagulans, Bacillus pseudomycoides and Bacillus lentus. In a preferred embodiment, the superoxide dismutase is not originating from E. coli.

Concentrates: The term “concentrates” means feed with high protein and energy concentrations, such as fish meal, molasses, oligosaccharides, sorghum, seeds and grains (either whole or prepared by crushing, milling, etc. from e.g. corn, oats, rye, barley, wheat), oilseed press cake (e.g. from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

Feed efficiency: The term “feed efficiency” means the amount of weight gain per unit of feed when the animal is fed ad-libitum or a specified amount of food during a period of time. By “increased feed efficiency” it is meant that the use of a feed additive composition according the present invention in feed results in an increased weight gain per unit of feed intake compared with an animal fed without said feed additive composition being present.

Feed Conversion Ratio (FCR): FCR is a measure of an animal's efficiency in converting feed mass into increases of the desired output. Animals raised for meat—such as swine, poultry and fish—the output is the mass gained by the animal. Specifically, FCR is calculated as feed intake divided by weight gain, all over a specified period. Improvement in FCR means reduction of the FCR value. An FCR improvement of 2% means that the FCR was reduced by 2%.

Feed Premix: The incorporation of the composition of feed additives as exemplified herein above to animal feeds, for example poultry feeds, is in practice carried out using a concentrate or a premix. A premix designates a preferably uniform mixture of one or more microingredients with diluent and/or carrier. Premixes are used to facilitate uniform dispersion of microingredients in a larger mix. A premix according to the invention can be added to feed ingredients or to the drinking water as solids (for example as water soluble powder) or liquids.

European Production Efficiency Factor (EPEF): The European Production Efficiency Factor is a way of comparing the performance of animals. This single-figure facilitates comparison of performance within and among farms and can be used to assess environmental, climatic and animal management variables. The EPEF is calculated as [(liveability (%)×Liveweight (kg))/(Age at depletion (days)×FCR)]×100, wherein livability is the percentage of animals alive at slaughter, Liveweight is the average weight of the animals at slaughter, age of depletion is the age of the animals at slaughter and FCR is the feed conversion ratio at slaughter.

Forage: The term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Fragment: The term “fragment” means a polypeptide or a catalytic domain having one or more (e.g., several) amino acids absent from the amino and/or carboxyl terminus of a mature polypeptide or domain; wherein the fragment has SOD activity.several) amino acids absent from the amino and/or carboxyl terminus of a mature polypeptide or domain; wherein the fragment has SOD activity.

Heat Stress: Heat stress occurs when an animal's heat load is greater than its capacity to lose heat. Pigs and other animals likely experience headaches, irritability and lethargy when they are too hot and have insufficient water. One or more of the following are typically observed with heat stress: increased breathing rate and sweating, increased water intake, decreased feed intake.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc.

Physical determination of the mature N terminus of SODs was done with Mass Spectrometry. Samples were diluted to 0.1 mg/ml in water. If they were to be deglycosylated before analysis, the samples were suspended in 50 mM Ammonium acetate buffer pH 5.5. The samples are then placed in an Ultimate 3000 UHPLC system (Thermo Scientific) at 8 degrees C. and run over an Advance Bio-RP desalting column (Agilent) The solvents used were A: LC/MS grade water with 0.1% formic acid, solvent: B 95% acetonitrile with 0.1% formic acid. The gradient was 5-80% B over 5 minutes. Post column the protein eluent was analyzed in a Bruker Maxis II mass spectrometer (Bremen Germany) and the resulting trace was analyzed by the supplied Bruker data analysis software. The deconvoluted spectrum was then compared to the calculated molecular weight with the expected N and C terminals using GPMAW (General Protein/Mass Analysis for Windows) software version 12.20. If the values match within 1 Dalton, a match was concluded.

Nutritional Stress: In pigs, poultry and other animals, symptoms of nutritional stress include impaired growth, immune suppression, reduced gut health, reduced gut integrity, shift in gut microflora and vomiting. In poultry, further symptoms include decreased egg production, decreased hatchability, gizzaed lesions, increased susceptibility to necrotic enteritis.

Obtained or obtainable from: The term “obtained or obtainable from” means that the polypeptide may be found in an organism from a specific taxonomic rank. If the taxonomic rank of a polypeptide is not known, it can easily be determined by a person skilled in the art by performing a BLASTP search of the polypeptide (using e.g. the National Center for Biotechnology Information (NCIB) website http://www.ncbi.nlm.nih.gov/) and comparing it to the closest homologues. The skilled person can also compare the sequence to those of the application as filed. An unknown polypeptide which is a fragment of a known polypeptide is considered to be of the same taxonomic species. An unknown natural polypeptide or artificial variant which comprises a substitution, deletion and/or insertion in up to 10 positions is considered to be from the same taxonomic species as the known polypeptide.

Oxidative Stress: The term “oxidative stress” is intended to mean an imbalance between oxidants and reductants (antioxidants) at the cellular or individual level. Oxidative damage is one result of such an imbalance and includes oxidative modification of cellular macromolecules, cell death by apoptosis or necrosis, as well as structural tissue damage by means of reactive oxygen and nitrogen species (ROS, RNS).

Roughage: The term “roughage” means dry plant material with high levels of fiber, such as fiber, bran, husks from seeds and grains and crop residues (such as stover, copra, straw, chaff, sugar beet waste).

Secreted Enzyme: A secreted enzyme is an exoenzyme, or extracellular enzyme, in that is is an enzyme that is secreted by a cell and functions outside that cell.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

$$( Identical Residues \times 100 ) / ( Length of Alignment - Total Number of Gaps in Alignment )$$

Substantially pure polypeptide: The term “substantially pure polypeptide” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the polypeptide is at least 92% pure, e.g., at least 94% pure, at least 95% pure, at least 96% pure, at least 97% pure, at least 98% pure, at least 99%, at least 99.5% pure, and 100% pure by weight of the total polypeptide material present in the preparation. The polypeptides of the present invention are preferably in a substantially pure form. This can be accomplished, for example, by preparing the polypeptide by well known recombinant methods or by classical purification methods.

Variant: The term “variant” means a polypeptide having SOD activity comprising an alteration, i.e., a substitution, insertion, and/or deletion, of one or more (several) amino acid residues at one or more (e.g., several) positions. A substitution means replacement of the amino acid occupying a position with a different amino acid; a deletion means removal of the amino acid occupying a position; and an insertion means adding 1, 2, or 3 amino acids adjacent to and immediately following the amino acid occupying the position.

In one aspect, a SOD variant may comprise from 1 to 10 alterations, i.e. 1, 2, 3, 4, 5, 6, 7, 8, 9 or 10 alterations and have at least 20%, e.g., at least 40%, at least 50%, at least 60%, at least 70%, at least 80%, at least 90%, at least 95%, or at least 100% of the SOD activity of the parent SOD, such as SEQ ID NO: 1 to 5.

Nutrient: The term “nutrient” in the present invention means components or elements contained in dietary feed for an animal, including water-soluble ingredients, fat-soluble ingredients and others. The example of water-soluble ingredients includes but is not limited to carbohydrates such as saccharides including glucose, fructose, galactose and starch; minerals such as calcium, magnesium, zinc, phosphorus, potassium, sodium and sulfur; nitrogen source such as amino acids and proteins, vitamins such as vitamin B1, vitamin B2, vitamin B3, vitamin B6, folic acid, vitamin B12, biotin and phatothenic acid. The example of the fat-soluble ingredients includes but is not limited to fats such as fat acids including saturated fatty acids (SFA); mono-unsaturated fatty acids (MUFA) and poly-unsaturated fatty acids (PUFA), fibre, vitamins such as vitamin A, vitamin E and vitamin K.

### Polypeptides of Bacterial Origin Having Superoxide Dismutase Activity

Polypeptides of bacterial origin have been found to have superoxide dismutase activity. These polypeptides are suitable for use in animal feed.

One aspect of the invention is directed to an isolated polypeptide having superoxide dismutase activity selected from the group consisting of:
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 17;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 19; and
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21.

Alternatively defined, an aspect of the invention is directed to an isolated polypeptide of having superoxide dismutase activity selected from the group consisting of
a. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 2;
b. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 4;
c. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 6;
d. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 8;
e. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 10;
f. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 12;
g. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 14;
h. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 16;
i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 18; and
j. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 20.

The polypeptide having superoxide dismutase activity is of bacterial origin and typically originating from a bacteria selected from the group consisting of a Bacillus species, a Neobacillus species, an Alkalihalobacillus species, a Pedobacter species, a Novosphingobium species, a Mongoliicoccus species, a Variovorax species, a Paraburkholderia species and a Arcicella aquatica species, most preferably a Bacillus species. Suitably, the said bacterial origin is selected from the group consisting of a Bacillus subtilis, Bacillus licheniformis, Bacillus cereus, Bacillus circulans, Bacillus coagulans, Bacillus pseudomycoides, Bacillus lentus, Neobacillus bataviensis, Alkalihalobacillus hwajinpoensis, Arcicella aquatica, Paraburkholderia sediminicola, Pedobacter nyackensis, Variovorax boronicumulans, Mongoliicoccus sp, Novosphingobium sp, and Pedobacter soli, preferably Bacillus subtilis, Bacillus licheniformis, Bacillus cereus, Bacillus circulans, Bacillus coagulans, Bacillus pseudomycoides and Bacillus lentus.

The polypeptide having superoxide dismutase activity is an EC 1.15.1.1 super oxide dismutase (SOD) of bacterial origin. Preferably, it is not of E. coli origin.

The polypeptide of having superoxide dismutase activity is typically selected from the group consisting of
a. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 2;
b. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 4;
c. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 6;
d. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 8;
e. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 10;
f. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 12;
g. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 14;
h. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 16;
i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 18; and
j. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 20.

### Method of Producing a Polypeptide Having Superoxide Dismutase

The polypeptides of the invention are made by recombinant methods. Accordingly, an aspect of the invention, is a method of producing a polypeptide having superoxide dismutase activity, comprising: cultivating a host cell under conditions suitable for expression of the polypeptide; and recovering the polypeptide. The host cell is a recombinant host cell transformed with the polynucleotide as defined herein. Accordingly, a further aspect of the invention is a recombinant process comprising a nucleotide of bacterial origin, encoding for a polypeptide having superoxide dismutase activity, said nucleotide selected from the group consisting of
a. polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 2;
b. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 4;
c. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 6;
d. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 8;
e. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 10;
f. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 12;
g. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 14;
h. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 16;
i. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 18; and
j. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 20.

A further aspect of the invention is directed to an isolated polynucleotide of bacterial origin encoding for a polypeptide having superoxide dismutase activity, wherein the polynucleotide is selected from the group consisting of
a. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 2;
b. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 4;
c. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 6;
d. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 8;
e. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 10;
f. polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 12;
g. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 14;
h. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 16;
i. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 18; and
j. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 20.

A further aspect of the invention is directed to a nucleic acid construct or expression vector comprising the polynucleotide of the invention. A related aspect of the invention is directed to a transgenic plant, plant part or plant cell transformed with the polynucleotide of the invention. Similarly, as an aspect of the invention is directed to a method of producing the polypeptide of the invention comprising: i. cultivating a transgenic plant or a plant cell comprising a polynucleotide encoding the polypeptide under conditions conducive for production of the polypeptide; and ii. recovering the polypeptide.

The invention is furthermore directed to whole broth formulation or cell culture composition comprising the polypeptide having superoxide dismutase activity as defined herein.

### Animal Feed and Animal Feed Additive

An important aspect of the invention is related to suitability of the polypeptides of the invention to be used in animal feed or as part of an animal feed additive. An animal feed additive comprising a polypeptide of bacterial origin having superoxide dismutase activity would benefit farmers and farm animals. The present invention also relates to the use of polypeptides of bacterial origin having superoxide dismutase activity in an animal feed or animal feed additive for improving any one of the performance parameter selected from the group consisting of
improving one or more performance parameters, wherein the one or more performance parameters is selected from the group consisting of the European Production Efficiency Factor (EPEF), Feed Conversion Ratio (FCR), Growth Rate (GR), Body Weight Gain (WG), Mortality Rate (MR) and Flock Uniformity (FU),
improving or enhancing immune response and/or reducing inflammation and/or for the modulation of the gut flora in an animal, and
reducing or eliminating the use of antibiotics administered to animal feed.

In one aspect, the animal feed and animal feed additive further comprise a catalase. The animal feed additive may comprise an enzyme component, wherein the enzyme component comprises all of the enzymes of the additive and the enzyme component consists of a superoxide dismutase of bacterial origin and optionally of a catalase. Alternatively defined, the animal feed additive may comprise at least one and no more than two enzyme classes, wherein the at least one enzyme class is a superoxide dismutase of bacterial origin and the optional second enzyme class is a catalase.

The animal feed additive or animal feed comprises a polypeptide of bacterial origin, suitably a polypeptide as defined above.

The animal feed or feed additive may further comprise one or more polypeptides having catalase activity and/or further comprise one or more vitamins, suitably the one or more vitamins is a fat-soluble vitamin, preferably vitamin E.

A related aspect is directed to a use of an animal feed additive as defined above as an antioxidant, preferably in feed and feed premixes.

In view of the properties of superoxide dismutases, a related aspect is directed to a use of an animal feed additive as defined above for replacing or partially replacing antibiotics in animal feed.

One aspect is directed to an animal feed comprising one or more protein sources and one or more energy sources characterized in that the animal feed further comprises one or more polypeptides of bacterial origin having superoxide dismutase (SOD) activity, wherein the animal feed
a. improves or enhances immune response; or
b. reduces inflammation;
when fed to the animal as compared to animals fed a feed composition without the polypeptides; and
said feed optionally further comprising one or more polypeptides having catalase activity.

In relation to the feed additive, it may be formulated in various forms as know to the person skilled in the art. Accordingly, an aspect is directed to a granule, which comprises:
a. a core comprising a polypeptide of bacterial origin having superoxide dismutase activity, typically comprising the polypeptide defined above, and, optionally
b. a coating consisting of one or more layer(s) surrounding the core.

Alternative, the feed additive may be a granule, which comprises:
c. a core, and
d. a coating consisting of one or more layer(s) surrounding the core, wherein the coating comprises a polypeptide of bacterial origin having superoxide dismutase activity, typically comprising the polypeptide defined above.

A further aspect is directed to an animal feed comprising one or more protein sources and one or more energy sources characterized in that the animal feed further comprises one or more polypeptides of bacterial origin having superoxide dismutase (SOD) activity, wherein said polypeptide is as defined above.

### A Method of Improving One or More Performance Parameters

The polypeptides of bacterial origin described herein when part of an animal feed or animal feed additive improve, according to an aspect of the invention, performance parameters in animals. Accordingly, the polypeptides allow for a method of improving one or more performance parameters in an animal comprising administering to the animal an animal feed or animal feed additive comprising one or more polypeptides having superoxide dismutase (SOD) activity of bacterial origin, wherein the one or more performance parameters is selected from the group consisting of the European Production Efficiency Factor (EPEF), Feed Conversion Ratio (FCR), Growth Rate (GR), Body Weight Gain (WG), Mortality Rate (MR) and Flock Uniformity (FU).

Accordingly, the polypeptides further allow for a method of improving or enhancing immune response and/or reducing inflammation and/or for the modulation of the gut flora in an animal comprising administering to the animal an animal feed or animal feed additive comprising one or more polypeptides having superoxide dismutase activity of bacterial origin.

The polypeptides further allow for a method of reducing or eliminating the use of antibiotics administered to an animal feed, comprising administering to the animal an animal feed or animal feed additive comprising of one or more polypeptides having superoxide dismutase activity of bacterial origin.

The polypeptide according to the method has superoxide dismutase activity, is of bacterial origin and typically originating from a bacteria selected from the group consisting of a Bacillus species, a Neobacillus species, an Alkalihalobacillus species, a Pedobacter species, a Novosphingobium species, a Mongoliicoccus species, a Variovorax species, a Paraburkholderia species and a Arcicella aquatica species, most preferably a Bacillus species. Suitably, the said bacterial origin is selected from the group consisting of a Bacillus subtilis, Bacillus licheniformis, Bacillus cereus, Bacillus circulans, Bacillus coagulans, Bacillus pseudomycoides, Bacillus lentus, Neobacillus bataviensis, Alkalihalobacillus hwajinpoensis, Arcicella aquatica, Paraburkholderia sediminicola, Pedobacter nyackensis, Variovorax boronicumulans, Mongoliicoccus sp, Novosphingobium sp, and Pedobacter soli, preferably Bacillus subtilis, Bacillus licheniformis, Bacillus cereus, Bacillus circulans, Bacillus coagulans, Bacillus pseudomycoides and Bacillus lentus.

The animal to which the animal feed or animal feed additive is fed according to the method, may have experienced heat stress, cold stress, nutritional stress and/or oxidative stress.

The method typically reduces cellular markers of reactive oxygen species or free radicals.

According to the method, the animal feed or animal feed additive may further comprise one or more polypeptides having catalase activity. The polypeptide having catalase activity is classified as an EC 1.11.1.6 catalase or as an EP 1.11.1.21 catalase peroxidase.

### PREFERRED EMBODIMENTS

1. An isolated polypeptide having superoxide dismutase activity selected from the group consisting of:
a. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
b. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3;
c. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5;
d. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7;
e. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9;
f. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
g. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 13;
h. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
i. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 17;
j. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 19; and
k. a polypeptide having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21.
2. An isolated polypeptide of having superoxide dismutase activity selected from the group consisting of
a. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 2;
b. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 4;
c. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 6;
d. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 8;
e. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 10;
f. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 12;
g. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 14;
h. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 16;
i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 18; and
j. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 20.
3. A nucleotide of bacterial origin, encoding for a polypeptide having superoxide dismutase activity, said nucleotide selected from the group consisting of
a. polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 2;
b. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 4;
c. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 6;
d. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 8;
e. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 10;
f. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 12;
g. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 14;
h. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 16;
i. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 18; and
j. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 20.
4. The polypeptide as defined in any of embodiments 1 and 2, said polypeptide being of bacterial origin.
5. The polynucleotide according to embodiment 3, said polynucleotide being of bacterial origin.
6. The polypeptide according to embodiment 4, wherein the said bacterial origin is selected from the group consisting of a Bacillus species, a Neobacillus species, an Alkalihalobacillus species, a Pedobacter species, a Novosphingobium species, a Mongoliicoccus species, a Variovorax species, a Paraburkholderia species and a Arcicella aquatica species.
7. The polynucleotide according to embodiment 5, wherein the said bacterial origin is selected from the group consisting of a Bacillus species, a Neobacillus species, an Alkalihalobacillus species, a Pedobacter species, a Novosphingobium species, a Mongoliicoccus species, a Variovorax species, a Paraburkholderia species and a Arcicella aquatica species.
8. The polypeptide according to embodiment 6, wherein the said bacterial origin is selected from the group consisting of a Bacillus subtilis, Bacillus licheniformis, Bacillus cereus, Bacillus circulans, Bacillus coagulans, Bacillus pseudomycoides, Bacillus lentus, Neobacillus bataviensis, Alkalihalobacillus hwajinpoensis, Arcicella aquatica, Paraburkholderia sediminicola, Pedobacter nyackensis, Variovorax boronicumulans, Mongoliicoccus sp, Novosphingobium sp, and Pedobacter soli.
9. The polynucleotide according to embodiment 5, wherein the said bacterial origin is selected from the group consisting of a Bacillus subtilis, Bacillus licheniformis, Bacillus cereus, Bacillus circulans, Bacillus coagulans, Bacillus pseudomycoides, Bacillus lentus, Neobacillus bataviensis, Alkalihalobacillus hwajinpoensis, Arcicella aquatica, Paraburkholderia sediminicola, Pedobacter nyackensis, Variovorax boronicumulans, Mongoliicoccus sp, Novosphingobium sp, and Pedobacter soli.
10. An isolated polypeptide of having superoxide dismutase activity selected from the group consisting of
a. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 2;
b. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 4;
c. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 6;
d. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 8;
e. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 10;
f. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 12;
g. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 14;
h. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 16;
i. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 18; and
j. a polypeptide encoded by a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 20.
11. An isolated polynucleotide of bacterial origin encoding for a polypeptide having superoxide dismutase activity, wherein the polynucleotide is selected from the group consisting of
   a. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 2;
   b. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 4;
   c. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 6;
   d. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 8;
   e. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 10;
   f. polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 12;
   g. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 14;
   h. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 16;
   i. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 18; and
   j. a polynucleotide having at least 80%, at least 85%, at least 90%, at least 91%, at least 92%, at least 93%, at least 94%, at least 95%, at least 96%, at least 97%, at least 98%, at least 99% or 100% sequence identity to the polypeptide coding sequence of SEQ ID NO 20.
12. A nucleic acid construct or expression vector comprising the polynucleotide of embodiment 11.
13. A recombinant host cell transformed with the polynucleotide of embodiment 11.
14. A method of producing a polypeptide having superoxide dismutase activity, comprising: cultivating the host cell of embodiment 13 under conditions suitable for expression of the polypeptide; and
   recovering the polypeptide.
15. A transgenic plant, plant part or plant cell transformed with the polynucleotide of embodiment 11.
16. A method of producing the polypeptide of any one of embodiments 1-11 comprising:
   i. cultivating a transgenic plant or a plant cell comprising a polynucleotide encoding the polypeptide under conditions conducive for production of the polypeptide; and
   ii. recovering the polypeptide.
17. A whole broth formulation or cell culture composition comprising the polypeptide of any one of embodiments 1-11.
18. An animal feed additive comprising a polypeptide of bacterial origin having superoxide dismutase activity.
19. The animal feed additive according to embodiment 18 further comprising a catalase.
20. The animal feed additive according to embodiment 18 comprising an enzyme component, wherein the enzyme component comprises all of the enzymes of the additive and the enzyme component consists of a superoxide dismutase of bacterial origin and optionally of a catalase.
21. An animal feed additive comprising at least one and no more than two enzyme classes, wherein the at least one enzyme class is a superoxide dismutase of bacterial origin and the optional second enzyme class is a catalase.
22. The animal feed additive according to any of embodiments 18 to 21 wherein the polypeptide having superoxide dismutase activity is as defined in any of embodiments 1 to 11.
22. The animal feed additive according to any of embodiments 18 to 22 wherein the feed additive further comprises one or more polypeptides having catalase activity and/or further comprising one or more vitamins.
23. The animal feed additive of embodiment 22, wherein the one or more vitamins is a fat-soluble vitamin, preferably vitamin E.
21. A method of improving one or more performance parameters in an animal comprising administering to the animal an animal feed or animal feed additive comprising one or more polypeptides having superoxide dismutase (SOD) activity of bacterial origin, wherein the one or more performance parameters is selected from the group consisting of the European Production Efficiency Factor (EPEF), Feed Conversion Ratio (FCR), Growth Rate (GR), Body Weight Gain (WG), Mortality Rate (MR) and Flock Uniformity (FU).
22. A method of improving or enhancing immune response and/or reducing inflammation and/or for the modulation of the gut flora in an animal comprising administering to the animal an animal feed or animal feed additive comprising one or more polypeptides having superoxide dismutase activity of bacterial origin.
23. A method of reducing or eliminating the use of antibiotics administered to an animal feed, comprising administering to the animal an animal feed or animal feed additive comprising of one or more polypeptides having superoxide dismutase activity of bacterial origin.
24. The method according to any of embodiments 21 to 23, wherein the animal has experienced heat stress, cold stress, nutritional stress and/or oxidative stress.
25. The method of embodiments 21 to 24 to reduce cellular markers of reactive oxygen species or free radicals.
26. The method of any of embodiments 21 to 25, wherein the polypeptide having superoxide dismutase activity is an EC 1.15.1.1 super oxide dismutase (SOD) of bacterial origin.
27. The method of any of embodiments 21 to 26, wherein the polypeptide of bacterial origin having superoxide dismutase activity is as defined in any of embodiments 1 to 11.
28. The method of any of embodiments 21 to 27, wherein the polypeptide having superoxide dismutase (SOD) activity of bacterial origin is selected from the group consisting of a Cu-SOD, a Zn-SOD, a Mn-SOD, and an Fe-SOD.
29. The method according to any of embodiments 21 to 28, wherein the animal feed or animal feed additive further comprises one or more polypeptides having catalase activity.
30. The method according to any of embodiments 18 to 29, wherein the polypeptide having catalase activity is classified as an EC 1.11.1.6 catalase or as an EP 1.11.1.21 catalase peroxidase.
32. Use of an animal feed additive as defined in any of embodiments 11 to 17 as an antioxidant, preferably in feed and feed premixes.
33. Use of an animal feed additive as defined in any of embodiments 11 to 17 for replacing or partially replacing antibiotics in animal feed.
34. An animal feed comprising one or more protein sources and one or more energy sources characterized in that the animal feed further comprises one or more polypeptides pf bacterial origin having superoxide dismutase (SOD) activity, wherein the animal feed
a. improves or enhances immune response; or
b. reduces inflammation;
when fed to the animal as compared to animals fed a feed composition without the polypeptides; and
said feed optionally further comprising one or more polypeptides having catalase activity.
36. A granule, which comprises:
   a. a core comprising the polypeptide of any one of embodiments 1 to 11, and, optionally
   b. a coating consisting of one or more layer(s) surrounding the core.
37. A granule, which comprises:
   a. a core, and
   b. a coating consisting of one or more layer(s) surrounding the core, wherein the coating comprises the polypeptide of any one of embodiments 1 to 11.
38. A composition comprising the polypeptide of any one of embodiments 1 to 11 or the granule of embodiment 35 and 36.
39. An animal feed comprising one or more protein sources and one or more energy sources characterized in that the animal feed further comprises one or more polypeptides of bacterial origin having superoxide dismutase (SOD) activity, wherein said polypeptide is as defined in any one of embodiments 1 to 11.

### EXAMPLES

### Example 1: Cloning and Expression of Polypeptides

The DNA encoding the SOD genes of embodiments of the invention, from bacterial strains isolated from different countries are listed in table 1.

TABLE 1

| SEQ ID NO | Donor | Country of Origin |
|---|---|---|
| SEQ ID NO: 1 | Bacillus sp-62775 | United States |
| SEQ ID NO: 3 | Bacillus sp-18780 | Denmark |
| SEQ ID NO: 5 | Bacillus sp-17964 | United States |
| SEQ ID NO: 7 | Bacillus sp-62802 | United States |
| SEQ ID NO: 9 | Variovorax boronicumulans | Germany |
| SEQ ID NO: 11 | Bacillus sp-18318 | Japan |
| SEQ ID NO: 13 | Pedobacter nyackensis | United States |
| SEQ ID NO: 15 | Mongoliicoccus sp-62519 | China |
| SEQ ID NO: 17 | Arcicella aquatica | United States |
| SEQ ID NO: 19 | Paraburkholderia sediminicola | Sweden |

The codon optimized synthetic DNA encoding the mature peptide sequences of the superoxide dismutases were ordered at the company TWIST Bioscience and cloned into the Bacillus expression vector described in WO 12/025577. Briefly, the DNA encoding the mature peptide of the SOD genes SEQ ID NO: 2, SEQ ID NO: 4 SEQ ID NO: 8 and SEQ ID NO: 10 were cloned in frame to a Bacillus clausii secretion signal, BcSP, with the following amino acid sequence: MKKPLGKIVASTALLISVAFSSSIASA (SEQ ID NO:23) whereas BcSP replaced the native secretion signal in the gene.

The DNA encoding the mature peptide of the SOD genes SEQ ID NO: 5 and SEQ ID NO:11 lacking a signal peptide in the natural protein, were cloned in the same vector without the BcSP Bacillus clausii secretion signal, to obtain intracellular expression.

Downstream of the SOD mature peptide sequences, an affinity tag sequence was introduced to ease the purification process (His-tag; with the following amino acid sequence: HHHHHH (SEQ ID NO: 24). The gene that was expressed therefore comprised the mature SOD sequence followed by the His tag sequence.

The final expression plasmids were transformed into a Bacillus subtilis expression host. The SOD genes were integrated by homologous recombination into the Bacillus subtilis host cell genome upon transformation.

The gene constructs were expressed under the control of a triple promoter system (as described in WO 99/43835). The gene coding for chloramphenicol acetyltransferase was used as maker (as described in (Diderichsen et al., 1993, Plasmid 30: 312-315)). Transformants were selected on LB media agar supplemented with 6 microgram of chloramphenicol per ml. One recombinant Bacillus subtilis clone containing each SOD expression construct was selected and was cultivated on a rotary shaking table in 500 ml baffled Erlenmeyer flasks each containing 100 ml yeast extract-based media. After 3-5 days cultivation time at 30° C. to 37° C., enzyme containing supernatants were harvested by centrifugation and the enzymes were purified by His-tag purification.

### Example 2: His Tag Purification Method

The His-tagged SOD enzymes were purified by immobilized metal chromatography (IMAC) using Ni2+ as the metal ion on 5 mL HisTrap Excel columns (GE Healthcare Life Sciences). The purification took place at pH 7 and the bound protein was eluted with imidazole. The purity of the purified enzymes was checked by SDS-PAGE and the concentration of t2e enzyme determined by Absorbance 280 nm after a buffer exchange in 50 mM HEPES, 100 mM NaCl pH7.0.

### Example 3—Superoxide Dismutase Activity Determination

### Principle:

Superoxide dismutases (SOD) catalyzes the reaction (dismutation) of superoxide anion into hydrogen peroxide and molecular oxygen. The activity is assayed as follow:

Superoxide-anions are formed enzymatically by xanthine oxidase from hypoxanthine. During the xanthine oxidase catalyzed reaction, hypoxanthine is transformed into hydrogen peroxide, uric acid and superoxide anions using atmospheric oxygen simultaneously. The water-soluble tetrazolium salt, WST-1, produces a water-soluble formazan dye upon reduction with a superoxide anion. The rate of WST-1 reduction to WST-1 formazan by superoxide anion is linearly related to the xanthine oxidase catalyzed superoxide anion formation and is inhibited by SOD. Absorbance at 450 nm is proportional to the amount of superoxide anion, the SOD activity as an inhibition activity can be quantified by measuring the decrease in the color development at 450 nm. Thus, the larger the activity of SOD, the slower the formation of the WST-1 formazan.

### Method

Mix 200 μl of a WST-1 working solution (1 mM WST-1 in 100 mM Tris-HCl, pH 8.0, 0.1 mM EDTA, 0.14 mM hypoxanthine, 0.0225% Brij) with 25 μl enzyme sample and 25 μl xanthine oxidase working solution (0,014 U/mL xanthine oxidase in 100 mM Tris-HCl, pH 8.0, 0.1 mM EDTA, 0.0225% Brij). Measure the change in absorption at 450 nm at a temperature of 30° C. for 30 minutes. In place of the enzyme sample, a buffer (blank) control is run, which will give maximum color development due to no inhibition from SOD activity.

### Data Evaluation:

The slope of the Absorption as a function of time is determined for each enzyme and all tested enzyme concentrations. A standard curve was recorded using an enzyme standard with a known specific activity and the standard curve was corrected for non-linearity by the following function:

$$y = {R}_{0} + {K}_{\text{?}} * \frac{1}{1 + exp ( - ( a + b * ln ( x ) )}$$

$$\text{?} \text{indicates text missing or illegible when filed}$$

Where y is the absorption of the standard for a known activity. The inverse of this formula is used to determine the activity for each enzyme and the slope of the so-obtained activity as a function of the enzyme concentration is calculated for each candidate, yielding the enzyme candidates specific activity. Then, the ratio of the slopes and the standard is calculated with the associated error as follows:

$$Error = \sqrt{{( \frac{Error of Enzyme candidate specific activity}{Enzyme candidate specific activity} )}^{2} + {( \frac{Error of Standard specific activity}{Standard specific activity} )}^{2}}$$

### Results

The table (Table 2) with the relative activity calculated relative to the commercially available SOD from Sigma (Commercially available bacterial (from E. coli) Mn-SOD (S5639 from Sigma))

TABLE 2

| | Relative | Donor Scientific |
|---|---|---|
| SOD name | activity | Name |
| Standard (Sigma SOD) | 100% | E. coli |
| SEQ ID NO: 1 | 1700% | Bacillus sp-62775 |
| SEQ ID NO: 3 | 1100% | Bacillus sp-18780 |
| SEQ ID NO: 5 | 800% | Bacillus sp-17964 |
| SEQ ID NO: 7 | 700% | Bacillus sp-62802 |
| SEQ ID NO: 9 | 600% | Variovorax boronicumulans |
| SEQ ID NO: 11 | 400% | Bacillus sp-18318 |
| SEQ ID NO: 13 | 400% | Pedobacter nyackensis |
| SEQ ID NO: 15 | 500% | Mongoliicoccus sp-62519 |
| SEQ ID NO: 17 | 400% | Arcicella aquatica |
| SEQ ID NO: 19 | 100% | Paraburkholderia sediminicola |
| SOD20-1 | 100% | Bacillus lentus |
| SOD32-1 | 0% | Neobacillus bataviensis |
| SOD34-1 | 200% | Bacillus sp-62615 |
| SOD19-1 | 0% | Alkalihalobacillus hwajinpoensis |
| SOD 7103 | 0% | Rheinheimera sp-64250 |
| SOD 7097 | 0% | Rhizobium nepotum |

The polypeptides of the invention were selected in that they outperform the commercially available bacterial SOD, from E. coli, available from Aldrich. As can be seen from the table Bacillus species performs well. A number of species did not perform well (not all data shown), as demonstrated by SODs at least from Alkalihalobacillus hwajinpoensis, Rheinheimera sp-64250, Rhizobium nepotum.

1. A nucleic acid construct comprising a heterologous polynucleotide that encodes a polypeptide having superoxide dismutase activity, said polypeptide selected from the group consisting of:
polypeptides having at least 75% sequence identity to SEQ ID NO: 1;
polypeptides having at least 75% sequence identity to SEQ ID NO: 3;
polypeptides having at least 75% sequence identity to SEQ ID NO: 5;
polypeptides having at least 75% sequence identity to SEQ ID NO: 7;
polypeptides having at least 75% sequence identity to SEQ ID NO: 9;
polypeptides having at least 75% sequence identity to SEQ ID NO: 11;
polypeptides having at least 75% sequence identity to SEQ ID NO: 13;
polypeptides having at least 75% sequence identity to SEQ ID NO: 15;
polypeptides having at least 75% sequence identity to SEQ ID NO: 17;
polypeptides having at least 75% sequence identity to SEQ ID NO: 19; and
polypeptides having at least 75% sequence identity to SEQ ID NO: 21.

2. The nucleic acid construct of claim 1, said polynucleotide selected from the group consisting of:
polynucleotides having at least 80% sequence identity to the polypeptide coding sequence of SEQ ID NO: 2;
polynucleotides having at least 80% sequence identity to the polypeptide coding sequence of SEQ ID NO: 4;
polynucleotides having at least 80% sequence identity to the polypeptide coding sequence of SEQ ID NO: 6;
polynucleotides having at least 80% sequence identity to the polypeptide coding sequence of SEQ ID NO: 8;
polynucleotides having at least 80% sequence identity to the polypeptide coding sequence of SEQ ID NO: 10;
polynucleotides having at least 80% sequence identity to the polypeptide coding sequence of SEQ ID NO: 12;
polynucleotides having at least 80% sequence identity to the polypeptide coding sequence of SEQ ID NO: 14;
polynucleotides having at least 80% sequence identity to the polypeptide coding sequence of SEQ ID NO: 16;
polynucleotides having at least 80% sequence identity to the polypeptide coding sequence of SEQ ID NO: 18; and
polynucleotides having at least 80% sequence identity to the polypeptide coding sequence of SEQ ID NO: 20.

3. The nucleic acid construct of claim 1, wherein said polypeptide is a copper-containing superoxide dismutase.

4. The nucleic acid construct of claim 1, wherein said polypeptide is a zinc-containing superoxide dismutase.

5. The nucleic acid construct of claim 1, wherein said polypeptide is a manganese-containing superoxide dismutase.

6. The nucleic acid construct of claim 1, wherein said polypeptide is an iron-containing superoxide dismutase.

7. A recombinant host cell comprising a heterologous polynucleotide that encodes a polypeptide having superoxide dismutase activity, said polypeptide selected from the group consisting of:
polypeptides having at least 75% sequence identity to SEQ ID NO: 1;
polypeptides having at least 75% sequence identity to SEQ ID NO: 3;
polypeptides having at least 75% sequence identity to SEQ ID NO: 5;
polypeptides having at least 75% sequence identity to SEQ ID NO: 7;
polypeptides having at least 75% sequence identity to SEQ ID NO: 9;
polypeptides having at least 75% sequence identity to SEQ ID NO: 11;
polypeptides having at least 75% sequence identity to SEQ ID NO: 13;
polypeptides having at least 75% sequence identity to SEQ ID NO: 15;
polypeptides having at least 75% sequence identity to SEQ ID NO: 17;
polypeptides having at least 75% sequence identity to SEQ ID NO: 19; and
polypeptides having at least 75% sequence identity to SEQ ID NO: 21.

8. The nucleic acid construct of claim 7, said polynucleotide selected from the group consisting of:
polynucleotides having at least 80% sequence identity to the polypeptide coding sequence of SEQ ID NO: 2;
polynucleotides having at least 80% sequence identity to the polypeptide coding sequence of SEQ ID NO: 4;
polynucleotides having at least 80% sequence identity to the polypeptide coding sequence of SEQ ID NO: 6;
polynucleotides having at least 80% sequence identity to the polypeptide coding sequence of SEQ ID NO: 8;
polynucleotides having at least 80% sequence identity to the polypeptide coding sequence of SEQ ID NO: 10;
polynucleotides having at least 80% sequence identity to the polypeptide coding sequence of SEQ ID NO: 12;
polynucleotides having at least 80% sequence identity to the polypeptide coding sequence of SEQ ID NO: 14;
polynucleotides having at least 80% sequence identity to the polypeptide coding sequence of SEQ ID NO: 16;
polynucleotides having at least 80% sequence identity to the polypeptide coding sequence of SEQ ID NO: 18; and
polynucleotides having at least 80% sequence identity to the polypeptide coding sequence of SEQ ID NO: 20.

9. The nucleic acid construct of claim 1, wherein said polypeptide is a copper-containing superoxide dismutase.

10. The nucleic acid construct of claim 1, wherein said polypeptide is a zinc-containing superoxide dismutase.

11. The nucleic acid construct of claim 1, wherein said polypeptide is a manganese-containing superoxide dismutase.

12. The nucleic acid construct of claim 1, wherein said polypeptide is an iron-containing superoxide dismutase.

13. A method of producing a polypeptide having superoxide dismutase activity, said method comprising cultivating the recombinant host cell of claim 1 under conditions suitable for expression of said polypeptide having superoxide dismutase activity.

14. The method of claim 13, further comprising recovering said polypeptide having superoxide dismutase activity.

15. A method of producing an animal feed additive, said method comprising incorporating a polypeptide having superoxide dismutase activity produced according to the method of claim 13 into a carrier to form an animal feed additive.

16. The method of claim 15, wherein said polypeptide having superoxide dismutase activity is incorporated into a granule comprising a core and one or more coating layers surrounding said core.

17. The method of claim 16, wherein said polypeptide having superoxide dismutase activity is incorporated into the core of said granule.

18. The method of claim 16, wherein said polypeptide having superoxide dismutase activity is incorporated into a coating layer of said granule.

19. An animal feed additive comprising a polypeptide having superoxide dismutase activity, said polypeptide selected from the group consisting of polypeptides having at least 80% identity to SEQ ID NO: 19 and polypeptides having at least 80% identity to SEQ ID NO: 21.

20. The animal feed additive of claim 19, further comprising a catalase.
